(19) United States
(12) Patent Application Publication
(10) Pub. No.: US 2014/0255320 A1
(43) Pub. Date: Sep. 11, 2014

(54) COSMETIC COMPOSITIONS

(71) Applicants: Christine Mendrok-Edinger, Basel (CH); Horst Westenfelder, Basel (CH)

(72) Inventors: Christine Mendrok-Edinger, Basel (CH); Horst Westenfelder, Basel (CH)

(73) Assignee: DSM IP ASSETS B.V., Heerlen (NL)

(21) Appl. No.: 14/124,677

(22) PCT Filed: Jun. 6, 2012
(86) PCT No.: PCT/EP2012/060663

§ 371 Date: May 21, 2014

(30) Foreign Application Priority Data
Jun. 8, 2011 (EP) 11169197.8

(51) Int. Cl. A61K 8/49; A61K 8/37; A61Q 17/04

(52) U.S. Cl. CPC A61K 8/496; A61Q 17/04; A61K 8/37
USPC 424/59; 514/785

(57) ABSTRACT

The present invention relates to a composition for topical application comprising at least a benzotriazol derivative and vitamin E or a derivative thereof. Furthermore, the invention relates to compositions that prevent staining of clothes.

The present invention relates to a composition for topical application comprising at least one paraben and at least a benzotriazol derivative.

To protect cosmetics against mold and bacteria, most cosmetic products currently on the market contain preservatives called parabens. While parabens protect against bacteria and fungi, studies have linked daily exposure to these substances to an increased risk of cancer and endocrine problems. Thus, many cosmetic manufactures are searching for alternatives which allow reducing the amount of parabens and don't appear to pose any health risks.

Surprisingly, it has been found that the use of specific benzotriazol derivatives in topical compositions reduces the microbiological growth and consequently allows the reduction of parabens to be used.

Thus, the invention relates in one aspect to a topical composition comprising at least one paraben and at least one benzotriazol derivative of formula (I)

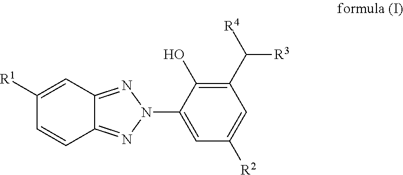

wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen;
characterized in that the benzotriazol derivative of formula (I) is present in an amount ranging from 1 to 20 wt.-% based on the total weight of the composition.

In another aspect the invention relates to the use of a benzotriazol derivative of formula (I) for increasing the antimicrobial activity, such as in particular the fungal activity of at least one paraben.

In yet a further embodiment the invention relates to a method of increasing the antimicrobial activity such as in particular the fungal activity of at least one paraben, said method comprising the addition of at least one benzotriazol derivative of formula (I) into said topical composition and observing or appreciating the result.

The term “Cₓ-C_{y}alkyl” as used herein refers to straight-chain or branched alkyl radicals having x to y carbon atoms such as e.g. methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methyl pentyl, 3-methylpentyl, 4-methylpentyl, 1,1-dimethylpropyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl without being limited thereto.

The term C₅₋₁₀cycloalkyl denotes to unsubstituted or C₁₋₁₀alkyl (mono- or poly-)substituted, in particular C₁₋₅alkyl (mono- or poly-)substituted cyclic, bicyclic or tricyclic hydrocarbon residues such as in particular cyclopentyl, cyclohexyl, cycloheptyl or decahydronaphtyl. Preferably, C₅₋₁₀cycloalkyl denotes to unsubstituted or C₁₋₂alkyl (mono- or poly-)substituted cyclopentyl, cyclohexyl or cycloheptyl such as in particular to unsubstituted or (mono- or poly-)methyl substituted cyclohexyl such as most in particular cyclohexyl or 3,3,5-trimethyl-cyclohexyl.

The term “Cₓ-C_{y}alkoxy” as used herein denotes to linear or branched alkoxy-, respectively unsubstituted or (mono- or poly-)substituted cycloalkoxy radicals having from x to y carbon atoms such as e.g. methoxy, ethoxy, propoxy, butyloxy or pentyloxy, 2,5,5-trimethylhexyloxy, 3,5,5-trimethylhexyloxy, isoamyloxy, 2-ethylhexyloxy or 3,3,5-trimethyl-cyclohexyloxy.

The term C₆₋₁₀aryl refers e.g. to naphthyl or phenyl radicals, preferably phenyl.

The term “paraben” refers to esters of para-hydroxybenzoic acid. Common parabens include methylparaben [CAS No. 99-76-3], ethylparaben [CAS No. 120-47-8], propylparaben [CAS No 94-13-3], butylparaben [CAS No. 94-26-8], heptylparaben [CAS1085-12-7], isobutylparaben [CAS No. 4247-02-3], isopropylparaben [CAS No. 4191-73-5] and benzylparaben [CAS No. 94-18-8] as well as salts thereof such as in particular sodium salts.

The term “antimicrobial” activity refers to the capability to destroy or inhibit the growth of microorganisms such as fungi or bacteria.

The (total) amount of the paraben in the topical compositions according to the present invention is not critical and advantageously selected in the range of 0.001 to 5 wt.-% such as in particular in the range of 0.0.01 to 1 wt.-% such as most in particular in the range of 0.01 to 0.5 wt.-%, based on the total weight of the composition.

Particular suitable parabenes according to the present invention are methylparabene, ethylparaben, butylparaben, isopropylparaben and/or propylparaben. These parabens are e.g. commercially available as Nipagin M, Nipagin A and Nipasol M or as a mixture of parabens with Phenoxyethanol as Phenonip (phenoxyethanol, methylparaben, ethylparaben, butylparaben, propylparaben, isobutylparaben) at Clariant UK Ltd.

Particularly suitable according to the present invention is a mixture of methyl-, ethyl- and propylparaben. Preferably, the methylparaben is used in an amount ranging from 0.1 to 0.3 wt.-% and the ethyl and propylparaben, independently of each other, are used in an amount of 0.02 to 0.1 wt.-%, based on the total weight of the composition.

Another suitable mixture of parabens is a mixture of methylparaben, ethylparaben, butylparaben, propylparaben, Isobutylparaben which furthermore comprises Phenoxyethanol which is commercially available as Phenonip. Phenonip is preferably used in an amount of 0.1 to 1.5 wt.-%, such as in particular of 0.25 to 1 wt.-%, based on the total weight of the composition.

The amount of the at least one benzotriazol derivative of formula (I) in the compositions according to the invention is preferable selected in the range of 2 to 20 wt.-%, such as in the range of 2 to 15 wt.-%, in particular in the range of 4 to 12 wt.-%, and most particular in the range of 4 to 10 wt.-% based on the total weight of the composition.

In a particular embodiment of the present invention the benzotriazol derivative is selected from compounds of formula (I) wherein R¹ and R⁴ are hydrogen, R² is methyl and R³ is C₅₋₁₀alkoxy such as preferably C₆₋₁₀alkoxy, or C₆cycloalkoxy such as in particular 2,5,5-trimethylhexyloxy, 3,5,5-trimethylhexyloxy, isoamyloxy, 2-ethylhexyloxy or 3,3,5-trimethyl-cyclohexyloxy. Such compounds and their preparation are e.g. disclosed in EP Application No.: 10150832.3 (PCT publication: WO2011/086124).

In another particular embodiment of the present invention the compound of formula (I) is a compound wherein R¹ and R⁴ are hydrogen, R² is methyl and R³ is undecyl (C₁₁ H₂₃) which is commercially available as Tinogard TL [INCI Name: benzotriazolyl dodecyl p-cresol; IUPAC Name: 2-(2H-benzotriazol-2-yl)-6-dodecyl-4-methyl-phenol] at BASF SE Ludwigshafen.

In another particular embodiment the compositions according to the present invention are substantially free of a polyglycerol based UV-filter such as e.g. disclosed in [EP Application No's] EP09178503.0, EP09178501.4, EP09178502.2 EP09178495.9, EP09178506.3, EP09178505.5 or EP10150832.3 which are obtainable by a process comprising the steps of ring-opening polymerization of x mol equivalents of glycidol using 1 mol equivalent of a polyol starter unit with y mol equivalents hydroxyl-groups, followed by block copolymerization with z×(x+y) mole equivalents of propylene oxide to form a hyperbranched polyether-polyol backbone carrying (x+y) mol equivalents hydroxyl-groups followed by partial or total esterification, respectively partial or total etherification of the hydroxyl groups with a UV-light absorbing chromophore such as particularly with p-dimethylamino benzoic acid, 3-[1-(4-Hydroxymethyl-phenyl)-meth-(E)-ylidene]-1,7,7-trimethyl-bicyclo[2.2.1]heptan-2-one, 2-(4-diethylamino-2-hydroxybenzoyl)benzoic acid, p-alkoxycinnamic acid, 2-cyano-3,3-diphenylacrylic acid as well as mixtures thereof and wherein x is an integer selected in the range from 3-16, y is an integer selected in the range from 1-6, and z is an integer selected in the range from 0-10.

The benzotriazol derivative of formula (I) with all the preferences as given herein is in particular effective to synergistically enhance the activity of the at least one paraben against the growth of fungi such as most in particular of Aspergillus brasiliensis and/or candida albicans.

The term “topical” is understood here to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, and keratinous fibres. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations, hair care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples for care preparations are hair-washing preparations in the form of shampoos, hair conditioners, hair-care preparations such as e.g. pretreatment preparations, hair tonics, styling creams, gels such as styling gels, pomades, hair rinses, treatment packs, intensive hair treatments, hair-straightening preparations, liquid hair-setting preparations, hair foams (hair mousses) and hairsprays.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are light-protective preparations, such as sun protection milks, sun protection lotions, sun protection creams, sun protection oils, sun blocks or tropical's or day care creams with a SPF (Sun Protection Factor). Of particular interest are sun protection creams, sun protection lotions, sun protection milks and sun protection preparations.

In another particular embodiment the topical compositions are hair-washing preparations in the form of shampoos or hair treatment preparations intended to be left in the hair (and not washed out) such as hair-setting preparations, hairsprays, gels, pomades, styling creams or hair foams (hair mousses), particularly hairsprays, gels or hair foams (hair mousses).

A shampoo may, for example, have the following composition: from 0.01 to 5 wt.-% of benzotriazol derivative of formula (I), 0.001 to 5 wt.-% of at least one paraben, 12.0 wt.-% of sodium laureth-2-sulfate, 4.0 wt.-% of cocamidopropyl betaine, 3.0 wt.-% of sodium chloride, and water ad 100 wt.-%.

The topical compositions according to the present invention may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

The topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples.

If the topical composition according to the invention is an O/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self emulsifying), stearic acid, salts of stearic acid, polyglyceryl-3-methylglycosedistearate. Further suitable emulsifiers are phosphate esters and the salts thereof such as cetyl phosphate (Amphisol® A), diethanolamine cetyl phosphate (Amphisol®DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one O/W respectively Si/W emulsifier is preferably used in an amount of 0.5 to 10 wt.-% such as in particular in the range of 0.5 to 5 wt.-% such as most in particular in the range of 1 to 4 wt.-% based on the total weight of the composition.

Particular suitable O/W emulsifiers according to the present invention encompass phosphate esters emulsifier of formula (II)

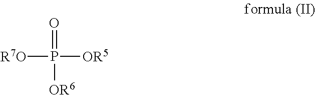

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. Particular advantageous phosphate ester emulsifier according to the invention are cetyl phosphates such as in particular potassium cetyl phosphate e.g. commercially available as Amphisol® K at DSM Nutritional Products Ltd Kaiseraugst.

Further suitable O/W emulsifiers are polyethyleneglycol (PEG) esters or diesters such as e.g. [INCI Names] (Glyceryl Stearate (and) PEG-100 Stearate), PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate. Particularly preferred according to the present invention is PEG-100 Stearate sold under the tradename Arlacel™ 165 (INCI Glyceryl Stearate (and) PEG-100 Stearate) by Croda.

Another particular suitable class of O/W emulsifiers are non ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

In particular embodiment, the invention relates to topical compositions in the form of O/W emulsions comprising an oily phase dispersed in an aqueous phase in the presence of an O/W emulsifier wherein the benzotriazol derivative of formula (I) is benzotriazolyl dodecyl p-cresol, the paraben is selected from the group consisting of methylparabene, ethylparaben, and propylparaben as well as mixtures thereof and the O/W emulsifier is selected from the group of cetyl phosphates such as particularly potassium cetyl phosphate.

In another particular embodiment, the invention relates to topical compositions in the form of O/W emulsions comprising an oily phase dispersed in an aqueous phase in the presence of an O/W emulsifier wherein the benzotriazol derivative of formula (I) is benzotriazolyl dodecyl p-cresol, the paraben is selected from the group consisting of methylparaben, ethylparaben, butylparaben, propylparaben, isobutylparaben as well as mixtures thereof and the O/W emulsifier is selected from the group of cetyl phosphates such as particularly potassium cetyl phosphate. Furthermore, it is advantageous if the composition further comprises phenoxyethanol. Particularly good results are obtained if the composition furthermore comprise a phenoxyethanol in an amount of about 0.01-3 wt.-% such as preferably in an amount of about 0.1 to 2 wt.-% such as most preferably in an amount of 0.5 to 1 wt.-%.

In a further embodiment, the invention relates to topical compositions in the form of O/W emulsions comprising an oily phase dispersed in an aqueous phase in the presence of an O/W emulsifier wherein the benzotriazol derivative of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy, the paraben is selected from the group consisting of methylparabene, ethylparaben, and propylparaben as well as mixtures thereof and the O/W emulsifier is selected from the group of cetyl phosphates such as particularly potassium cetyl phosphate.

In another particular embodiment, the invention relates to topical compositions in the form of O/W emulsions comprising an oily phase dispersed in an aqueous phase in the presence of an O/W emulsifier wherein the benzotriazol derivative of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy, the paraben is selected from the group consisting of methylparaben, ethylparaben, butylparaben, propylparaben, isobutylparaben as well as mixtures thereof and the O/W emulsifier is selected from the group of cetyl phosphates such as particularly potassium cetyl phosphate. Furthermore, it is advantageous if the composition further comprises phenoxyethanol. Particularly good results are obtained if the composition furthermore comprise a phenoxyethanol in an amount of about 0.01-3 wt.-% such as preferably in an amount of about 0.1 to 2 wt.-% such as most preferably in an amount of 0.5 to 1 wt.-%.

The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16) cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

The compositions in form of O/W emulsions according to the invention can be provided, for example, in all the formulation forms for O/W emulsions, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

Finally, a subject-matter of the invention is a method for the cosmetic treatment of keratinous substances such as in particular the skin, characterized in that a composition as defined above is applied to the said keratinous substances such as in particular to the skin. The method is in particular suitable to protect the skin against the adverse effects of UV-radiation such as in particular sun-burn and/or photoageing.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992), The Cosmetic, Toiletry and Fragrance Association, Inc. without being limited thereto.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions. Suitable further UV-filter substance to be incorporated into the compositions according to the present invention are conventional UVA and/or UVB and/or broad spectrum UV-filter substances known to be added into topical compositions such as cosmetic or dermatological sun care products. Such UV-filter substances comprise all groups which absorb light in the range of wavelengths 400 nm to 320 nm (UVA) and 320 nm to 280 nm (UVB) or of even shorter wavelengths (UVC) and which are or can be used as cosmetically acceptable UV-filter substances. Such UV-filter substances are e.g. listed in the CTFA Cosmetic ingredient Handbook or “The Encyclopedia of Ultraviolet Filters” (ISBN: 978-1-932633-25-2) by Nadim A. Shaath.

Suitable UV-filter substances may be organic or inorganic compounds. Exemplary organic UV-filter substances encompass e.g. acrylates such as e.g. 2-ethylhexyl 2-cyano-3,3-diphenylacrylate (octocrylene, PARSOL® 340), ethyl 2-cyano-3,3-diphenylacrylate; Camphor derivatives such as e.g. 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, terephthalylidene dicamphor sulfonic acid (Mexoryl® SX); Cinnamate derivatives such as e.g. ethylhexyl methoxycinnamate (PARSOL® MCX), ethoxyethyl methoxycinnamate, isoamyl methoxycinnamate as well as cinnamic acid derivatives bond to siloxanes; p-Aminobenzoic acid derivatives such as e.g. p-aminobenzoic acid, 2-ethylhexyl p-dimethylaminobenzoate, N-oxypropylenated ethyl p-aminobenzoate, glyceryl p-aminobenzoate; Benzophenones such as e.g. benzophenone-3, benzophenone-4,2,2′,4,4′-tetrahydroxy-benzophenone, 2,2′-dihydroxy-4,4′-dimethoxybenzophenone; Esters of benzalmalonic acid such as e.g. di-(2-ethylhexyl) 4-methoxybenzalmalonate; Organosiloxane compounds carrying chromophore groups such as e.g. polysilicones-15 (PARSOL® SLX), drometrizole trisiloxane (Mexoryl® XL); Imidazole derivatives such as e.g. 2-phenyl benzimidazole sulfonic acid (PARSOL®HS) and salts thereof such as e.g. sodium- or potassium salts, ammonium salts, morpholine salts, salts of primary, sec. and tert. amines like monoethanolamine salts, diethanolamine salts; Salicylate derivatives such as e.g. isopropylbenzyl salicylate, benzyl salicylate, butyl salicylate, ethylhexyl salicylate (PARSOL® EHS, Neo Heliopan® OS), isooctyl salicylate or homomenthyl salicylate (homosalate, PARSOL® HMS, Neo Heliopan® HMS); Triazine derivatives such as e.g. ethylhexyl triazone (Uvinul® T-150), diethylhexyl butamido triazone (Uvasorb® HEB), bis-ethylhexyloxyphenol methoxyphenyl triazine (Tinosorb® 5); Benzotriazole derivatives such as e.g. 2,2′-methylene-bis-(6-(2H-benzotriazole-2-yl)-4-(1,1,3,3,-tetramethylbutyl)-phenol (Tinosorb® M); Encapsulated UV-filters such as e.g. encapsulated ethylhexyl methoxycinnamate (Eusolex® UV-pearls) or microcapsules loaded with UV-filters as e.g. dislosed in EP 1471995; Phenylene-1,4-bis-benzimidazolsulfonic acids or salts such as e.g. 2,2-(1,4-phenylene)bis-(1H-benzimidazol-4,6-disulfonic acid) (Neoheliopan AP); Benzoxazol-derivatives such as e.g. 2,4-bis-[5-1(dimethylpropyl)benzoxazol-2-yl-(4-phenyl)-imino]-6-(2-ethylhexyl)-imino-1,3,5-triazin [Uvasorb® K2A); Dibenzoylmethane derivatives such as e.g. Butyl Methoxy Dibenzoylmethane; IUPAC Name: 1-(4-Methoxyphenyl)-3-(4-tert-butylphenyl)propane-1,3-dione) which is e.g. commercially available as PARSOL® 1789 at DSM Nutritional Products Ltd and amino substituted benzophenones such as Diethylamino hydroxybenzoyl hexyl benzoate [IUPAC Name: Hexyl 2-[4-(diethylamino)-2-hydroxybenzoyl]benzoate] sold under the tradename Uvinul® A plus by BASF. Inorganic UV-filter substances encompass pigments such as e.g. microparticulated Zink oxide or Titanium dioxide (e.g. commercially available as PARSOL® TX) The term “microparticulated” refers to a particle size from about 5 nm to about 200 nm, particularly from about 15 nm to about 100 nm. The particles may also be coated by other metal oxides such as e.g. aluminum or zirconium oxides or by silica or by organic coatings such as e.g. polyols, methicone, aluminum stearate, alkyl silane. Such coatings are well known in the art.

In order to enhance the photostability of sun care products it may be desirable to add a photostabilizer. Exemplary photostabilizers known to a skilled person in the art encompass e.g. 3,3-diphenylacrylate derivatives such as e.g. octocrylene (PARSOL® 340) or Polyester-8 (Polycrylene®) or Methoxycrylene (Solastay S1®); Benzylidene camphor derivatives such as e.g. 4-methyl benzylidene camphor (PARSOL® 5000); Benzalmalonate derivatives such as e.g. polysilicones-15 (PARSOL® SLX) or diethylhexyl syringylidene malonate (Oxynex ST liquid); Dialkyl naphthalates such as diethylhexyl naphthalate (Corapan TQ) without being limited thereto. An overview on further stabilizers is e.g. given in ‘SPF Boosters & Photostability of Ultraviolet Filters’, HAPPI, October 2007, p. 77-83 which is included herein by reference. The photostabilizers are generally used in an amount of 0.05 to 10 wt.-% with respect to the total weigh of the topical composition.

Generally, the amount of each UV-filter substance in the compositions according to the invention is selected in the range of about 0.1 to 10 wt.-%, preferably in the range of about 0.2 to 7 wt.-%, most preferably in the range of about 0.5 to 5 wt.-% with respect to the total weigh of the topical composition.

The total amount of UV-filter substances in the compositions according to the invention is preferably in the range of about 1 to 40 wt.-%, preferably in the range of about 5 to 30 wt.-%, in particular in the range of 10 to 30 wt.-% with respect to the total weight of the topical composition.

Preferred UVB-filter substances according to the invention encompass polysilicones-15, phenylbenzimidazol sulfonic acid, octocrylene, ethylhexyl methoxycinnamate, ethylhexyl triazone, ethyl hexylsalicylate, 4-methyl benzylidene camphor, benzophenones-3 and/or homosalate.

Preferred broadband UV-filter substances according to the invention encompass unsymmetrical s-triazine derivatives such 2,4-Bis-{[4-(2-ethyl-hexyloxy)-2-hydroxy]-phenyl}-6-(4-methoxyphenyl)-1,3,5-triazin, certain benzophenones such as e.g. 2-Hydroxy-4-methoxy-benzophenon, 2,2′-Methylen-bis-(6-(2H-benzotriazol-2-yl)-4-(1,1,3,3-tetramethyl-butyl)-phenol), and/or titanium dioxide.

Preferred UVA-filter substances encompass Butyl Methoxy Dibenzoylmethane and Diethylamino hydroxybenzoyl hexyl benzoate.

It is particularly suitable, if the topical compositions according to the present invention are sun care preparations comprising at least one, preferably at least two further UV-filter substances. Preferably the additional UV filter substances are selected from the group consisting of butyl methoxydibenzoylmethane polysilicone-15, octocrylene and phenylbenzimidazol sulfonic acid as well as mixtures thereof, most preferably butyl methoxydibenzoylmethane polysilicone-15, octocrylene and phenylbenzimidazol sulfonic acid are present. Preferably, butyl methoxydibenzoylmethane is used in an amount ranging from 1 to 5 wt.-%, polysilicone-15 in an amount of 1 to 10 wt.-%, octocrylene in an amount of 2 to 10 wt and phenylbenzimidazol sulfonic acid in an amount of 1 to 5 wt.-%, based on the total weight of the composition. Preferably the sun care preparations are O/W emulsions comprising an oily phase dispersed in an aqueous phase in the presence of an O/W emulsifier and the O/W emulsifier is potassium cetylphosphate, preferably used in an amount ranging from 1 to 5 wt.-%.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH according to standard methods in the art.

The following examples are provided to further illustrate the compositions and effects of the present invention. These examples are illustrative only and are not intended to limit the scope of the invention in any way.

### EXAMPLE

TABLE 1
O/W Emulsion without Benzotriazolyl Dodecyl p-Cresol (Reference)

| Phase | Ingredients | INCI Name | % w/w |
|---|---|---|---|
| A | Estol 3650 | Glyceryl Myristate | 4.00 |
| | Lanette 16 | Cetyl Alcohol | 2.00 |
| | Butylated Hydroxytoluene | BHT | 0.05 |
| | Dow Corning 200/100 cs | Dimethicone | 2.00 |
| | AMPHISOL ® K | Potassium Cetyl | 3.00 |
| | | Phosphate | |
| | Paraffin Oil | Mineral Oil | 3.60 |
| | PARSOL ® 1789 | Butyl | 4.00 |
| | | Methoxydibenzoylmethane | |
| | | (Avobenzone; USAN) | |
| | PARSOL ® SLX | Polysilicone-15 | 3.00 |
| | DUB DIS | Diisopropyl Sebacate | 12.00 |
| | Cetiol B | Dibutyl Adipate | 10.00 |
| | Antaron V-220 | VP/Eicosene Copolymer | 2.00 |
| | PARSOL ® 340 | Octocrylene | 3.60 |
| | | (Octocrilene; USAN) | |
| | Nipagin A | Ethylparaben | 0.05 |
| B | Glycerin | Glycerin | 3.00 |
| | Keltrol | Xanthan Gum | 0.30 |
| | Water dem. | Aqua | 29.50 |
| | Dequest 2046 | Pentasodium | 0.50 |
| | | Ethylenediamine | |
| | | Tetramethylene | |
| | | Phosphonate | |
| | Nipagin M | Methylparaben | 0.20 |
| | Nipasol M | Propylparaben | 0.05 |
| C | Sodium Hydroxide 10% | Sodium Hydroxide | 0.65 |
| | sol. | | |
| D | Water dem. | Aqua | 10.00 |
| | Triethanolamine (T.E.A.) | Triethanolamine | 2.50 |
| | PARSOL ® HS | Phenylbenzimidazole | 4.00 |
| | | Sulfonic Acid | |
| | | (Ensulizole; USAN) | |

### Procedure

1 Heat part A to 85° C. and stir until homogeneous.
3 Heat part B to 85° C. and add to part A under agitation, then add part C.
4 When everything is homogenous add part D pre-heated to 50° C.
   Be sure that the pH of PARSOL® HS solution is 7.0. If traces remain, add small quantities of the used neutralizing base until the particles are dissolved. Homogenize thoroughly and then with continued mixing cool down to ambient temperature.

TABLE 2
O/W Emulsion with Benzotriazolyl Dodecyl p-Cresol (example
according to present invention)

| Phase | Ingredients | INCI Name | % w/w |
|---|---|---|---|
| A | Estol 3650 | Glyceryl Myristate | 4.00 |
| | Lanette 16 | Cetyl Alcohol | 2.00 |
| | Butylated Hydroxytoluene | BHT | 0.05 |
| | Dow Corning 200/100 cs | Dimethicone | 2.00 |
| | AMPHISOL ® K | Potassium Cetyl Phosphate | 3.00 |
| | Paraffin Oil | Mineral Oil | 3.60 |
| | PARSOL ® 1789 | Butyl | 2.00 |
| | | Methoxydibenzoylmethane | |
| | | (Avobenzone; USAN) | |
| | PARSOL ® SLX | Polysilicone-15 | 3.00 |
| | DUB DIS | Diisopropyl Sebacate | 19.40 |
| | Cetiol B | Dibutyl Adipate | 10.00 |
| | Antaron V-220 | VP/Eicosene Copolymer | 2.00 |
| | PARSOL ® 340 | Octocrylene | 3.60 |
| | | (Octocrilene; USAN) | |
| | Tinogard TL | Benzotriazolyl | 10.00 |
| | | Dodecyl p-Cresol | |
| B | Glycerin | Glycerin | 3.00 |
| | Keltrol | Xanthan Gum | 0.30 |
| | Water dem. | Aqua | 24.00 |
| | Dequest 2046 | Pentasodium | 0.50 |
| | | Ethylenediamine | |
| | | Tetramethylene | |
| | | Phosphonate | |
| | Nipagin A | Ethylparaben | 0.05 |
| | Nipagin M | Methylparaben | 0.20 |
| | Nipasol M | Propylparaben | 0.05 |
| C | Sodium Hydroxide 10% | Sodium Hydroxide | 0.65 |
| | sol. | | |
| D | Triethanolamine (T.E.A.) | Triethanolamine | 0.60 |
| | Water dem. | Aqua | 5.00 |
| | PARSOL ® HS | Phenylbenzimidazole | 1.00 |
| | | Sulfonic Acid | |
| | | (Ensulizole; USAN) | |

### Procedure

1 Heat part A to 85° C. and stir until homogeneous.
2 Heat part B to 85° C. and add to part A under agitation, then add part C.
4 When everything is homogenous add part D pre-heated to 50° C.
   Be sure that the pH of PARSOL® HS solution is 7.0. If traces remain, add small quantities of the used neutralizing base until the particles are dissolved. Homogenize thoroughly and then with continued mixing cool down to ambient temperature.

The formulations were tested at UFAG Laboratorien AG, Kornfeldstrasse 4, CH-6210 Sursee for the Efficacy of Antimicrobial Preservation, Testing according to: Ph. Eur. 7 (2011) 5.1.3.-2. Category: preparation for cutaneous application, Storage Temperature all cultures 20-25° C.; Sample quantity of the single test: each 20 g; Inoculation rate: 1:100; Inactivation medium: SANARO; Method germ count: plate count medium.

The read out has been performed after 2 weeks. The results are presented in table 3.

TABLE 3

| | 1 (Reference) | 2 |
|---|---|---|
| Aspergillus brasiliensis | 270000 | 160000 |
| Candida albicans | 29000 | 18500 |

As can be retrieved from the results presented in table 1, the use of a benzotriazol derivative according to the present invention in combination with at least one paraben significantly reduces the microbiological growth.

1. A topical composition comprising at least on paraben and at least one benzotriazol derivative of formula (I) 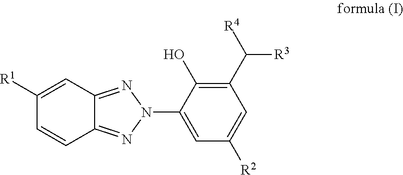
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen
characterized in that the benzotriazol derivative of formula (I) is present in an amount ranging from 1 to 20 wt.-% based on the total weight of the composition.

2. The topical composition according to claim 1, characterized in that the total amount of parabens is selected in the range of 0.001 to 5 wt.-% based on the total weight of the composition.

3. The topical composition according to claim 1, characterized in that the benzotriazol derivative is used in an amount selected in the range of 2 to 20 wt.-% based on the total weight of the composition.

4. The topical composition according to claim 1, characterized in that the benzotriazol compound of formula (I) is a compound wherein R¹ and R⁴ are hydrogen, R² is methyl and R³ is 2,5,5-trimethylhexyloxy, 3,5,5-trimethylhexyloxy, isoamyloxy, 2-ethylhexyloxy, 3,3,5-trimethyl-cyclohexyloxy or undecyl.

5. The topical composition according to claim 1, characterized in that the at least one paraben is selected fro the group consisting of methylparaben, ethylparaben, butylparaben, isopropylparaben and propylparaben as well as mixtures thereof.

6. The topical composition according to claim 1, characterized in that the at least one paraben is a mixture of methylparaben, ethylparaben, propylparaben.

7. The topical composition according to claim 1, characterized in that the topical composition is an O/W emulsion comprising an oily phase dispersed in an aqueous phase in the presence of an O/W emulsifier.

8. The topical composition according to claim 7, characterized in that the O/W emulsifier is selected from the group consisting of phosphate ester emulsifiers.

9. The topical composition according to claim 7, characterized in that the amount of O/W emulsifier is selected in the range of 0.5 to 10 wt.-%, based on the total weight of the composition.

10. The topical composition according to claim 1, characterized in that the composition comprises at least one co-surfactant in an amount selected in the range of 0.1 to 10 wt.-% based on the total weight of the composition.

11. The topical composition according to claim 10, characterized in that the co-surfactant is selected from the group consisting of cetyl alcohol, cetearyl alcohol, stearyl alcohol, behenyl alcohol, glyceryl stearate, glyceryl Myristate and hydrogenated coco-glycerides as well as mixtures thereof.

12. The topical composition according to claim 1, characterized in that the composition comprises at least one further UV-filter substance.

13. The topical composition according to claim 12, wherein the at least one UV-filter substance is selected from the group consisting of butyl methoxydibenzoylmethane polysilicone-15, octocrylene and phenylbenzimidazol sulfonic acid as well as mixtures thereof.

14. The topical composition according to claim 1, characterized in that the topical composition is a skin care preparation, hair care preparation, decorative preparation or a functional preparation.

15. Use of a benzotriazol derivative of formula (I) 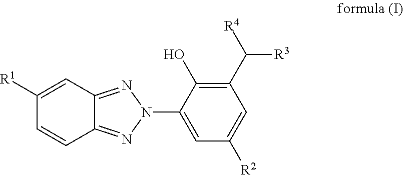
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl; C₅₋₁₀cycloalkyl; C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl; preferably hydrogen
for increasing the antimicrobial activity of at least one paraben.

16. Method of increasing the antimicrobial activity of at least one paraben in a topical composition, said method comprising the addition of at least one benzotriazol derivative of formula (I) 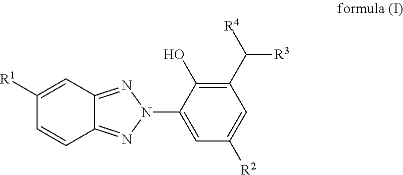
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloalkyl; C₁₋₂₀alkoxy or C₅₋₁₀cycloalkoxy; preferably C₅₋₁₅alkyl or C₅₋₁₅alkoxy; and
R⁴ is hydrogen or C₁₋₅alkyl; preferably hydrogen
into said topical composition and observing or appreciating the result.

17. A method for the cosmetic treatment of keratinous substances, characterized in that a composition as defined in claim 1 is applied to the said keratinous substances.
